(19) United States
(12) Patent Application Publication
(10) Pub. No.: US 2023/0295580 A1
(43) Pub. Date: Sep. 21, 2023

(54) SPHEROID GENERATOR, SPHEROID CULTURE KIT, AND SPHEROID CULTURE METHOD

(71) Applicant: RESEARCH AND BUSINESS FOUNDATION SUNGKYUNKWAN UNIVERSITY, Suwon-si (KR)

(72) Inventors: Sungsu PARK, Seoul (KR); Seok Gyu HAN, Yongin-si (KR)

(73) Assignee: RESEARCH AND BUSINESS FOUNDATION SUNGKYUNKWAN UNIVERSITY, Suwon-si (KR)

(21) Appl. No.: 18/019,754

(22) PCT Filed: Jun. 11, 2021
(86) PCT No.: PCT/KR2021/007344

§ 371 Date: Feb. 3, 2023

(30) Foreign Application Priority Data
Aug. 4, 2020 (KR) 10-2020-0097382

(51) Int. Cl. C12N 5/09; C12M 3/00; C12M 1/12; C12M 1/26; C12M 1/32

(52) U.S. Cl. CPC C12N 5/0693; C12M 21/08; C12M 25/01; C12M 33/04; C12M 23/12; C12N 2513/00

(57) ABSTRACT

A spheroid generator includes a body part; a main injection part which is formed in the body part so that a cancer cell solution is injected and configured to stop the flow of the cancer cell solution when the cancer cell solution comes into contact with a culture medium; and a sub-injection part which is formed in the body part so that an anticancer substance or a stromal cell solution is injected, and communicates with the main injection part so that the injected anticancer substance or stromal cell solution flows to the main injection part.

### TECHNICAL FIELD

The present invention relates to a spheroid generator, a spheroid culture kit, and a method of culturing a spheroid, and more specifically, to a spheroid generator, which is capable of easily manufacturing a spheroid and preventing a spheroid from falling to one side from a main injection part, a spheroid culture kit, and a method of culturing a spheroid.

### BACKGROUND ART

In general, cancer cells can be obtained from resected cancer tissue or blood of cancer patients. In the treatment of cancer patients, it is essential to culture cancer cells obtained from cancer patients to develop anticancer drugs that do not harm normal cells while suppressing the growth of cancer cells. As cancer cells are cultured, cancer spheroids, which are cancer masses, are produced.

However, conventionally, it was difficult to additionally add drugs or stromal cells to a cancer cell incubator. In order to inject drugs or stromal cells into a cancer cell incubator, the previously contained culture medium needs to be removed with a pipette and then reinjected into the same channel. Therefore, the time required to inject drugs or stromal cells was significantly increased, and there was a high possibility that spheroids would fall out of a cancer cell incubator due to the injection pressure of the drugs or stromal cells. Also, since there is no reservoir in a cancer cell incubator, the amount of sample that can be injected was limited.

The background art of the present invention is disclosed in Korean Patent Application Publication No. 2013-0122510 (published on Nov. 7, 2013, title of the invention: Cancer cell culture chip).

### DISCLOSURE

### Technical Problem

The present invention is directed to providing a spheroid generator, which is capable of easily manufacturing a spheroid and preventing a spheroid from falling to one side from a main injection part, a spheroid culture kit, and a method of culturing a spheroid.

### Technical Solution

A spheroid generator according to the present invention includes: a body part; a main injection part which is formed in the body part so that a cancer cell solution is injected and configured to stop the flow of the cancer cell solution when the cancer cell solution comes into contact with a culture medium; and a sub-injection part which is formed in the body part so that an anticancer substance or a stromal cell solution is injected and communicates with the main injection part so that the injected anticancer substance or stromal cell solution flows to the main injection part.

The main injection part may include: a main inlet part into which the cancer cell solution is injected and which communicates with the sub-injection part; and a main tube which communicates with the main inlet part and is configured to stop the flow of the cancer cell solution when the cancer cell solution comes into contact with the culture medium.

The main tube may include: a first main tube which is connected to one side of the main inlet part and formed to have an inner diameter smaller than that of the main inlet part; and a second main tube which is connected to one side of the first main tube and formed to have a diameter larger than that of the first main tube.

The body part may further include a communication channel part formed so that one side of the main inlet part and one side of the sub-injection part communicate with each other.

One sub-injection part may be formed around the main inlet part.

A plurality of sub-injection parts may be formed around the main inlet part.

The plurality of sub-injection parts may be formed to have the same inner diameter.

The plurality of sub-injection parts may be formed to have different inner diameters.

The body part, the main injection part, and the sub-injection part may be manufactured by 3D printing.

A spheroid culture kit according to the present invention include: a culture tank in which a culture medium is accommodated; a culture plate which is disposed at one side of the culture tank and in which a plurality of culture chambers are formed; and a plurality of spheroid generators respectively disposed in the plurality of culture chambers, wherein the spheroid generator includes: a body part accommodated in the culture chamber; a main injection part which is formed in the body part so that a cancer cell solution is injected and configured to stop the flow of the cancer cell solution when the cancer cell solution comes into contact with a culture medium; and a sub-injection part which is formed in the body part so that an anticancer substance or a stromal cell solution is injected and communicates with the main injection part so that the injected anticancer substance or stromal cell solution flows to the main injection part.

The main injection part may include: a main inlet part into which the cancer cell solution is injected and which communicates with the sub-injection part; and a main tube which communicates with the main inlet part and is configured to stop the flow of the cancer cell solution when the cancer cell solution comes into contact with a culture medium.

The main tube may include: a first main tube which is connected to one side of the main inlet part and formed to have an inner diameter smaller than that of the main inlet part; and a second main tube which is connected to one side of the first main tube and formed to have a diameter larger than that of the first main tube.

The body part may further include a communication channel part formed so that one side of the main inlet part and one side of the sub-injection part communicate with each other.

One sub-injection part may be formed around the main inlet part.

A plurality of sub-injection parts may be formed around the main inlet part.

A plurality of culture chambers may be formed in the form of a matrix in the culture plate, and the plurality of spheroid generators may be arranged in the form of a matrix in the culture plate.

In the culture plate, the sub-injection part may gradually increase in size from the spheroid generator at one side of the culture plate to the spheroid generator at the other side.

In the plurality of spheroid generators, main injection parts having the same size may be formed.

The spheroid generator may be manufactured by 3D printing.

A method of culturing a spheroid according to the present invention includes: disposing a spheroid generator in which the size of a sub-injection part is increased from one side to the other side of a culture plate; injecting a cancer cell solution into a main injection part of each of a plurality of spheroid generators; forming a spheroid as cancer cells of the cancer cell solution are cultured in the plurality of spheroid generators; injecting an anticancer substance into the sub-injection part of each of the plurality of spheroid generators; changing a cancer cell proportion of the spheroid according to an input amount of the anticancer substance in the plurality of spheroid generators; and injecting a culture medium into the main injection part of the plurality of spheroid generators so that the spheroid drops from the main injection part.

The step of forming a spheroid as cancer cells of the cancer cell solution are cultured in the plurality of spheroid generators may be performed for 12 to 36 hours.

The step of changing a cancer cell proportion of the spheroid according to the anticancer substance may be performed for 12 to 36 hours.

A method of culturing a spheroid according to the present invention includes: disposing a spheroid generator in which the diameter of a sub-injection part is increased from one side to the other side of a culture plate; injecting a cancer cell solution into a main injection part of each of a plurality of spheroid generators; forming a spheroid as cancer cells of the cancer cell solution are cultured in the plurality of spheroid generators; injecting a stromal cell solution into the sub-injection part of each of the plurality of spheroid generators; culturing a spheroid in which the proportions of the cancer cell and stromal cell are different according to an injected amount of the stromal cell solution in the plurality of spheroid generators; and injecting a culture medium into the main injection part of each of the plurality of spheroid generators so that the spheroid drops from the main injection part.

The cancer cell solution may include a cancer cell labeled with a first fluorescent solution.

The stromal cell solution may include a stromal cell labeled with a second fluorescent solution.

### Advantageous Effects

According to the present invention, since a sub-injection part is formed separately from a main injection part in a body part, an anticancer substance or a stromal cell solution can be injected into the sub-injection part.

In addition, according to the present invention, since an anticancer substance or a stromal cell solution can be injected through a sub-injection part without removing a culture medium in a main injection part, the time required to inject the anticancer substance or stromal cell solution can be substantially reduced. Also, an injected amount of the anticancer substance or stromal cell solution can be appropriately adjusted.

Additionally, according to the present invention, when the anticancer substance or stromal cell solution is injected, a spheroid of a cancer cell solution can be prevented from falling to one side from a main injection part due to injection pressure.

### DESCRIPTION OF DRAWINGS

FIG. 1 is a schematic perspective view of a spheroid generator according to an embodiment of the present invention.

FIG. 2 is a schematic cross-sectional view of a spheroid generator according to an embodiment of the present invention.

FIG. 3 is a schematic plan view of one example of a spheroid generator according to an embodiment of the present invention.

FIG. 4 is a schematic plan view of another example of a spheroid generator according to an embodiment of the present invention.

FIG. 5 is a schematic plan view of still another example of a spheroid generator according to an embodiment of the present invention.

FIG. 6 is a schematic cross-sectional view showing a state in which a cancer cell solution is injected into a main injection part in a spheroid generator according to an embodiment of the present invention.

FIG. 7 is a schematic cross-sectional view showing a state in which a cancer cell solution flows toward one side in a main injection part in a spheroid generator according to an embodiment of the present invention.

FIG. 8 is a schematic cross-sectional view showing a state in which an anticancer substance or a stromal cell solution is injected into a sub-injection part in a spheroid generator according to an embodiment of the present invention.

FIG. 9 is a schematic cross-sectional view showing a state in which a culture medium is injected into a main injection part in a spheroid generator according to an embodiment of the present invention.

FIG. 10 is a schematic plan view of a spheroid culture kit according to an embodiment of the present invention.

FIG. 11 is a schematic flowchart of one example of a spheroid culturing method according to an embodiment of the present invention.

FIG. 12 is a schematic diagram of cancer cells killed due to an anticancer substance and living cancer cells in a spheroid culturing method according to an embodiment of the present invention.

FIG. 13 is a graph schematically showing a relationship between an injected amount of an anticancer substance and cancer cell viability in a spheroid culturing method according to an embodiment of the present invention.

FIG. 14 is a schematic flowchart of another example of a spheroid culturing method according to an embodiment of the present invention.

FIG. 15 is a schematic diagram of stromal cells and cancer cells in a spheroid culturing method according to an embodiment of the present invention.

FIG. 16 is a graph schematically showing a relationship between an injected amount of stromal cells and cancer cells in a spheroid culturing method according to an embodiment of the present invention.

### MODES OF THE INVENTION

Hereinafter, embodiments of a spheroid generator, spheroid culture kit, and spheroid culturing method according to the present invention will be described with reference to the accompanying drawings. In describing a spheroid generator, a spheroid culture kit, and a spheroid culturing method, the thickness of lines, the size of components, and the like shown in the drawings may be exaggerated for clarity and convenience of description. Also, terms to be described below have been defined in consideration of their functions in the present invention and may have different meanings depending on the intention or custom of the user or worker. Therefore, the meanings of these terms should be interpreted based on the contents throughout the present specification.

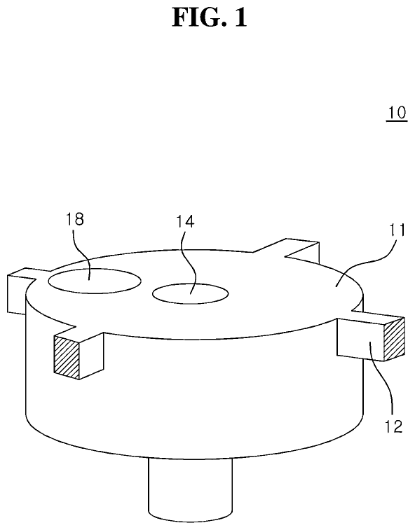

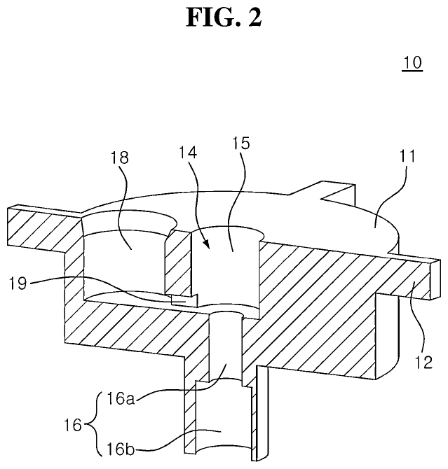

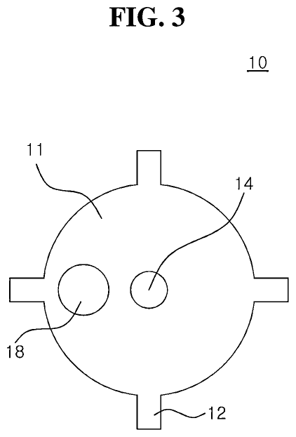

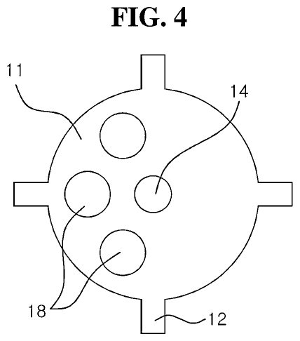

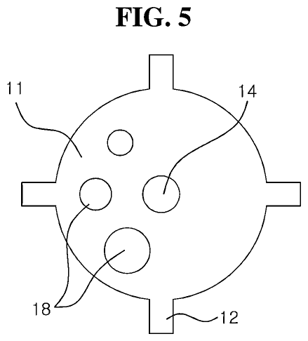

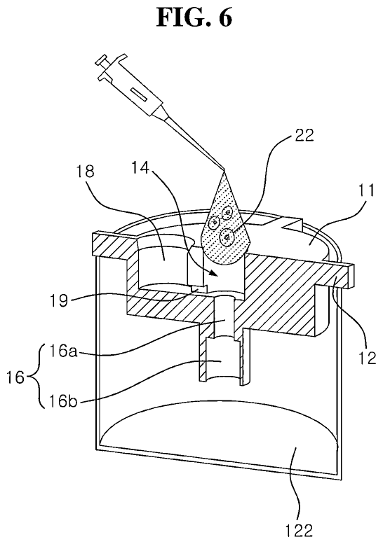

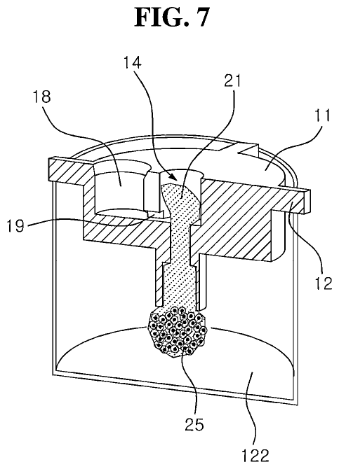

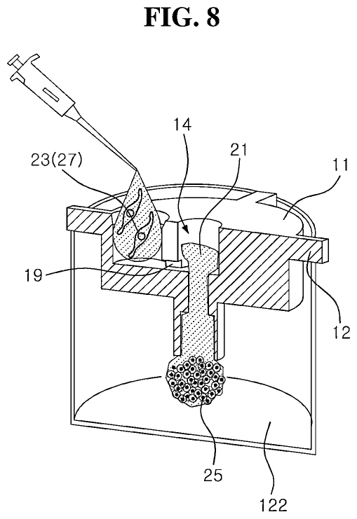

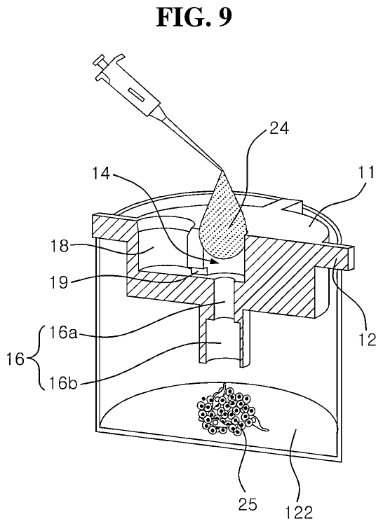

FIG. 1 is a schematic perspective view of a spheroid generator according to an embodiment of the present invention, FIG. 2 is a schematic cross-sectional view of a spheroid generator according to an embodiment of the present invention, FIG. 3 is a schematic plan view of one example of a spheroid generator according to an embodiment of the present invention, FIG. 4 is a schematic plan view of another example of a spheroid generator according to an embodiment of the present invention, FIG. 5 is a schematic plan view of still another example of a spheroid generator according to an embodiment of the present invention, FIG. 6 is a schematic cross-sectional view showing a state in which a cancer cell solution is injected into a main injection part in a spheroid generator according to an embodiment of the present invention, FIG. 7 is a schematic cross-sectional view showing a state in which a cancer cell solution flows toward one side in a main injection part in a spheroid generator according to an embodiment of the present invention, FIG. 8 is a schematic cross-sectional view showing a state in which an anticancer substance or a stromal cell solution is injected into a sub-injection part in a spheroid generator according to an embodiment of the present invention, and FIG. 9 is a schematic cross-sectional view showing a state in which a culture medium is injected into a main injection part in a spheroid generator according to an embodiment of the present invention.

Referring to FIGS. 1 to 9, a spheroid generator 10 according to an embodiment of the present invention includes a body part 11, a main injection part 14, and a sub-injection part 18.

The body part 11 may be made of a synthetic resin. The body part 11 may be formed in a cylindrical or polygonal columnar shape.

A plurality of support ribs 12 are formed at a circumference of the body part 11 so that the body part is seated in a culture chamber 122 of a culture plate 120. The support rib 12 may be radially formed at a circumference of the body part 11.

The main injection part 14 is formed in the body part 11 so that a cancer cell solution 21 is injected and configured to stop the flow of the cancer cell solution 21 when the cancer cell solution 21 comes into contact with a culture medium 24. In this case, the cancer cell solution 21 maintains a hanging state from the main injection part 14 due to self-aggregation. The main injection part 14 may be formed in a cylindrical shape as a whole.

The sub-injection part 18 is formed in the body part 11 so that an anticancer substance 23 or a stromal cell solution 27 is injected, and communicates with the main injection part 14 so that the injected anticancer substance 23 or stromal cell solution 27 flows to the main injection part 14. The sub-injection part 18 may be formed in a cylindrical shape.

Since the sub-injection part 18 is formed separately from the main injection part 14 in the body part 11, the anticancer substance 23 or the stromal cell solution 27 may be additionally injected into the sub-injection part 18. Also, since the anticancer substance 23 or the stromal cell solution 27 may be additionally injected through the sub-injection part 18 without removing the culture medium 24 in the main injection part 14, the time required to inject the anticancer substance 23 or the stromal cell solution 27 may be substantially reduced. Also, an injected amount of the anticancer substance 23 or stromal cell solution 27 may be appropriately adjusted.

In addition, since the sub-injection part 18 is formed in the body part 11, even when the anticancer substance 23 or the stromal cell solution 27 is injected into the sub-injection part 18, the injection pressure of the anticancer substance 23 or the stromal cell solution 27 is not applied to the main injection part 14. Therefore, when the anticancer substance 23 or the stromal cell solution 27 is injected, a spheroid 25 of the cancer cell solution 21 may be prevented from falling to one side from the main injection part 14 due to the injection pressure. Therefore, the spheroid 25 may be cultured while hanging from the main injection part 14. In this case, the spheroid 25 means a cancer mass in which cancer cells included in the cancer cell solution 21 are cultured similarly to the human body.

The main injection part 14 includes a main inlet part 15 and a main tube 16.

The cancer cell solution 21 is injected into the main inlet part 15, and the main inlet part 15 communicates with the sub-injection part 18. The main tube 16 communicates with the main inlet part 15 and is configured to stop the flow of the cancer cell solution 21 when the cancer cell solution 21 comes into contact with the culture medium 24. When the cancer cell solution 21 enters the main inlet part 15, the cancer cell solution 21 flows down through the main tube 16. In this case, when one side of the cancer cell solution 21 comes into contact with the culture medium 24 accommodated in the culture chamber 122, the cancer cell solution 21 no longer flows down in the main tube 16 due to self-aggregation of the cancer cell solution 21. Therefore, cancer cells positioned at one side of the cancer cell solution 21 may be cultured in a hanging state from the cancer cell solution 21. Furthermore, as cancer cells are cultured, the spheroid 25 may be cultured in the form of a cancer mass similar to the human body.

The main tube 16 includes: a first main tube 16a which is connected to one side of the main inlet part 15 and formed to have an inner diameter smaller than that of the main inlet part 15; and a second main tube 16b which is connected to one side of the first main tube 16a and formed to have a diameter larger than that of the first main tube 16a. Since the first main tube 16a is formed to have a diameter smaller than that of the second main tube 16b, as the frictional force of the cancer cell solution 21 increases in the first main tube 16a, the cancer cell solution 21 may maintain a hanging state in the first main tube 16a.

The body part 11 further includes a communication channel part 19 formed so that one side of the main inlet part 15 and one side of the sub-injection part 18 communicate with each other. The communication channel part 19 may have a circular cross section or a polygonal cross section. Therefore, the anticancer substance 23 or the stromal cell solution 27 injected into the sub-injection part 18 enters the main inlet part 15 through the communication channel part 19 and thus may be mixed with the cancer cell solution 21. Therefore, although the culture of cancer cells in the cancer cell solution 21 may be suppressed by the anticancer substance 23, the cancer cells may be cultured with stromal cells.

One sub-injection part 18 is formed around the main inlet part 15 (see FIG. 3). Therefore, one type of anticancer substance 23 or stromal cell solution 27 may be supplied to the main injection part 14 through the sub-injection part 18.

A plurality of sub-injection parts 18 are formed around the main inlet part 15 (see FIGS. 4 and 5). Therefore, multiple types of anticancer substances 23 or stromal cell solutions 27 may be supplied to the main injection part 14 through the plurality of sub-injection parts 18. Accordingly, the effect of a combination of multiple types of anticancer substances 23 or stromal cell solutions 27 on cancer cells can be confirmed.

The plurality of sub-injection parts 18 are formed to have the same inner diameter (see FIG. 4). Therefore, since multiple types of anticancer substances 23 or stromal cell solutions 27 are injected in the same amount into the sub-injection parts 18, the effect of the same amount of a combination of heterogeneous anticancer substances 23 or a combination of heterogeneous stromal cell solutions 27 on cancer cells can be confirmed.

The plurality of sub-injection parts 18 are formed to have different inner diameters (see FIG. 5). Therefore, since multiple types of anticancer substances 23 or stromal cell solutions 27 are injected in different amounts into the sub-injection parts 18, the effect of different amounts of a combination of heterogeneous anticancer substances 23 or a combination of heterogeneous stromal cell solutions 27 on cancer cells can be confirmed.

The body part 11, the main injection part 14, and the sub-injection part 18 may be manufactured by 3D printing. Therefore, the spheroid generator may be manufactured to have a miniaturized structure or a complex structure.

Next, a spheroid culture kit according to an embodiment of the present invention will be described.

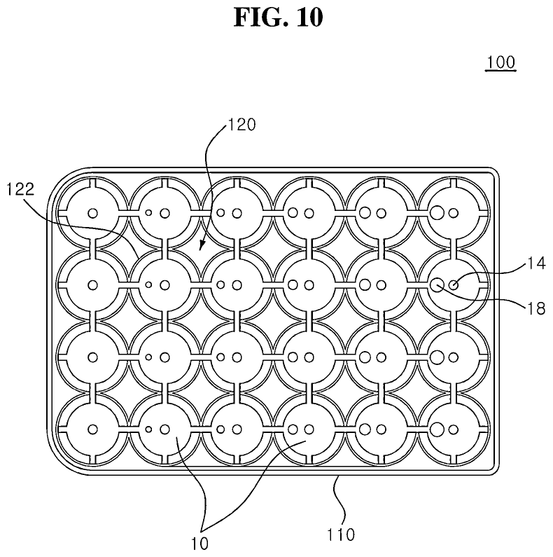

FIG. 6 is a schematic cross-sectional view showing a state in which a cancer cell solution is injected into a main injection part in a spheroid generator according to an embodiment of the present invention, FIG. 7 is a schematic cross-sectional view showing a state in which a cancer cell solution flows toward one side in a main injection part in a spheroid generator according to an embodiment of the present invention, FIG. 8 is a schematic cross-sectional view showing a state in which an anticancer substance or a stromal cell solution is injected into a sub-injection part in a spheroid generator according to an embodiment of the present invention, FIG. 9 is a schematic cross-sectional view showing a state in which a culture medium is injected into a main injection part in a spheroid generator according to an embodiment of the present invention, and FIG. 10 is a schematic plan view of a spheroid culture kit according to an embodiment of the present invention.

Referring to FIGS. 6 to 10, a spheroid culture kit 100 according to an embodiment of the present invention includes a culture tank 110, a culture plate 120, and a plurality of spheroid generators 10.

A culture medium 24 is accommodated in the culture tank 110. The culture tank 110 may be formed in the form of a quadrangular container. The culture plate 120 is disposed at one side of the culture tank 110 and has a plurality of culture chambers 122 formed therein. The culture plate 120 is formed in a quadrangular shape so as to cover one side of the culture tank 110. The plurality of spheroid generators 10 are respectively disposed in the plurality of culture chambers 122.

The plurality of culture chambers 122 are formed in the form of a matrix in the culture plate 120, and the plurality of spheroid generators 10 are arranged in the form of a matrix in the culture plate 120. Since the plurality of spheroid generators 10 are arranged in the form of a matrix, different types of anticancer substances 23 or stromal cell solutions 27 are injected into the sub-injection parts 18 of the spheroid generators 10, and accordingly, the effect on cancer cells can be confirmed.

In the culture plate 120, the sub-injection part 18 gradually increases in size from the spheroid generator 10 at one side of the culture plate to the spheroid generator 10 at the other side. In this case, when the size of the sub-injection part 18 is small, the amount of anticancer cells or stromal cells injected into the sub-injection part 18 is reduced. On the other hand, when the size of the sub-injection part 18 is large, the amount of anticancer cells or stromal cells injected into the sub-injection part 18 is increased. Therefore, when different amounts of anticancer substances 23 are supplied to cancer cells through the sub-injection part 18, a decrease in cancer cell viability according to the volume of the anticancer substance 23 can be confirmed. Also, when different amounts of stromal cell solutions are supplied to cancer cells through the sub-injection part 18, a plurality of spheroids 25 in which the proportions of cancer cells and stromal cells are different may be formed.

In the plurality of spheroid generators 10, main injection parts 14 having the same size are formed. Therefore, the cancer cell solution 21 injected through the main injection part 14 is injected in the same volume into all of the spheroid generators 10.

In the spheroid generator 10, a plurality of sub-injection parts 18 are formed around the main inlet part 15 (see FIGS. 4 and 5). Therefore, in the spheroid generator 10, multiple types of anticancer substances 23 or stromal cell solutions 27 may be supplied to the main injection part 14 through the plurality of sub-injection parts 18. Accordingly, the effect of a combination of multiple types of anticancer substances 23 or stromal cell solutions 27 on cancer cells can be confirmed.

In the spheroid generator 10, the plurality of sub-injection parts 18 are formed to have the same inner diameter (see FIG. 4). Therefore, in the spheroid generator 10, since multiple types of anticancer substances 23 or stromal cell solutions 27 are injected in the same amount into the sub-injection part 18, the effect of the same amount of a combination of heterogeneous anticancer substances 23 or a combination of heterogeneous stromal cell solutions 27 on cancer cells can be confirmed.

In the spheroid generator 10, the plurality of sub-injection parts 18 are formed to have different inner diameters (see FIG. 5). Therefore, in the spheroid generator 10, since multiple types of anticancer substances 23 or stromal cell solutions 27 are injected in different amounts into the sub-injection part 18, the effect of different amounts of a combination of heterogeneous anticancer substances 23 or a combination of heterogeneous stromal cell solutions 27 on cancer cells can be confirmed.

The spheroid generator 10 is manufactured by 3D printing. Therefore, the spheroid generator 10 may be manufactured to have a more precise and finer structure.

Since the configurations of the body part 11, main injection part 14, and sub-injection part 18 in the above-described spheroid generator 10 are substantially the same as those described above, the same reference numerals are assigned to the same configurations, and descriptions thereof will be omitted.

Next, a method of culturing a spheroid according to an embodiment of the present invention will be described.

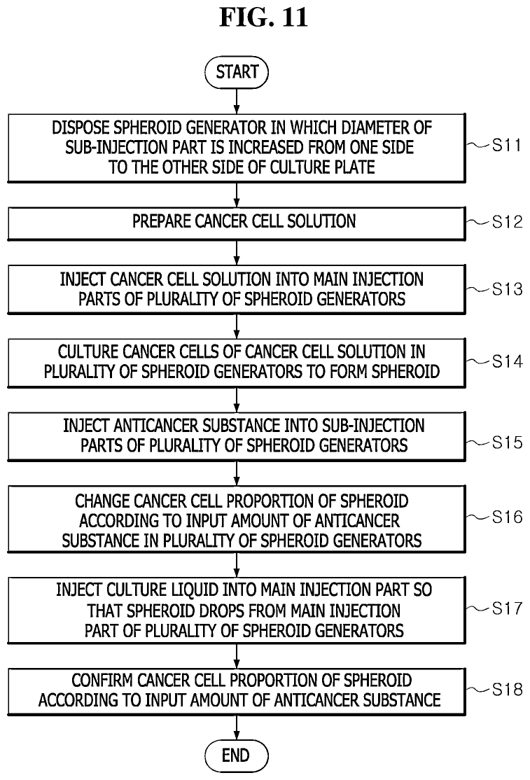

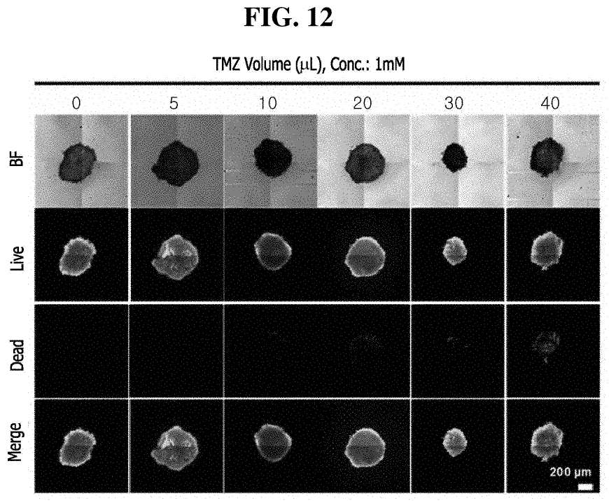

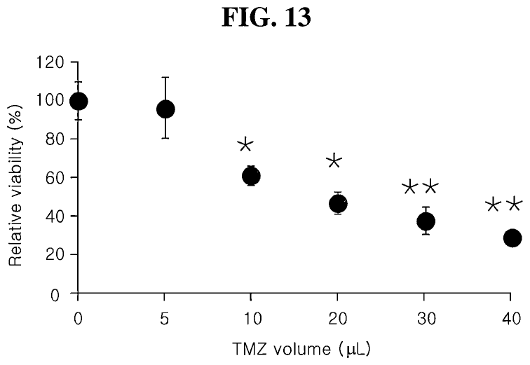

FIG. 11 is a schematic flowchart of one example of a spheroid culturing method according to an embodiment of the present invention, FIG. 12 is a schematic diagram of cancer cells killed due to an anticancer substance and living cancer cells in a spheroid culturing method according to an embodiment of the present invention, and FIG. 13 is a graph schematically showing a relationship between an amount of an anticancer substance injected and cancer cell viability in a spheroid culturing method according to an embodiment of the present invention.

Referring to FIG. 11 to FIG. 13, a spheroid generator 10 in which the size of a sub-injection part 18 is increased from one side to the other side of a culture plate 120 is disposed (S11). In this case, main injection parts 14 having the same size are formed in all of the spheroid generators 10.

A cancer cell solution 21 is prepared (S12). That is, the cancer cell solution 21 is prepared by mixing a plurality of cancer cells with a culture medium 24.

The cancer cell solution 21 is injected into the main injection parts 14 of the plurality of spheroid generators 10 (S13). In this case, since main injection parts 14 having the same size are formed in all of the spheroid generators 10, the same amount of the cancer cell solution 21 is injected into all of the main injection parts 14.

As the cancer cells of the cancer cell solution 21 are cultured in the plurality of spheroid generators 10, a spheroid 25 is formed (S14). In this case, the step of forming a spheroid 25 as the cancer cells of the cancer cell solution 21 are cultured in the plurality of spheroid generators 10 is performed for 12 to 36 hours.

An anticancer substance 23 is injected into a sub-injection part 18 of the plurality of spheroid generators 10 (S15). In this case, since the size of the sub-injection part 18 is increased from one side to the other side of a culture plate 120, an input amount (volume) of the anticancer substance 23 is increased from the sub-injection part 18 at one side of the culture plate toward the sub-injection part 18 at the other side.

A cancer cell proportion of the spheroid 25 is changed according to an input amount of the anticancer substance 23 in the plurality of spheroid generators 10 (S16). A cancer cell proportion is decreased in a spheroid generator 10 with a large input amount of the anticancer substance 23, and a cancer cell proportion is increased in a spheroid generator 10 with a small input amount of the anticancer substance 23. In this case, the step of changing a cancer cell proportion of the spheroid 25 according to the anticancer substance 23 is performed for 12 to 36 hours.

A culture medium 24 is injected into the main injection part 14 of each of the plurality of spheroid generators 10 so that the spheroid 25 drops from the main injection part 14 (S17). In this case, the spheroid 25 drops from the main injection part 14 toward one side due to the injection pressure of the culture medium 24 injected into the main injection part 14.

A cancer cell proportion of the spheroid 25 according to an input amount of the anticancer substance 23 is confirmed (S18). In this case, a cancer cell proportion of the spheroid 25 can be confirmed using a microscope.

Referring to FIGS. 10 and 11, the volume of dead cancer cells in the spheroid 25 is shown in the Dead line, the volume of living cancer cells is shown in the Live line, and the mixed volume of dead cancer cells and living cancer cells is shown in the Merge line. It can be seen that relative viability of cancer cells decreases as the volume of the anticancer substance 23 (TMZ) increases.

Next, a method of culturing a spheroid according to an embodiment of the present invention will be described.

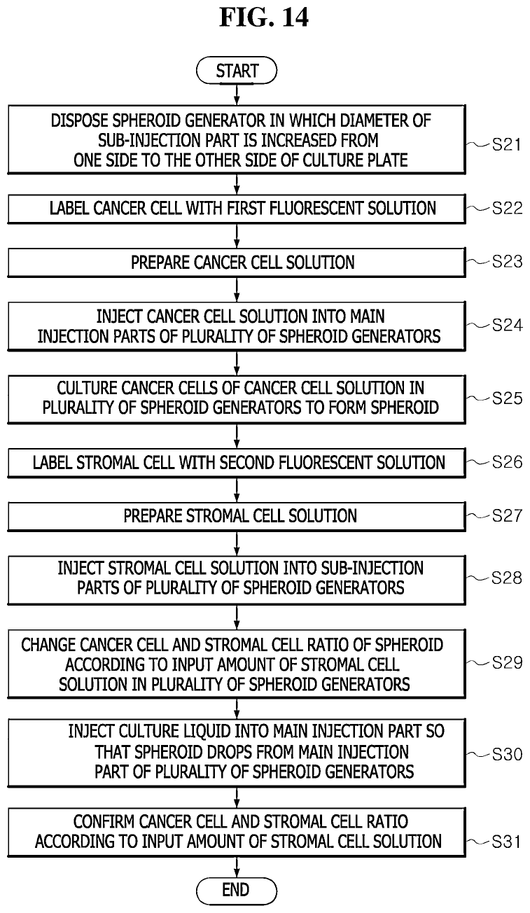

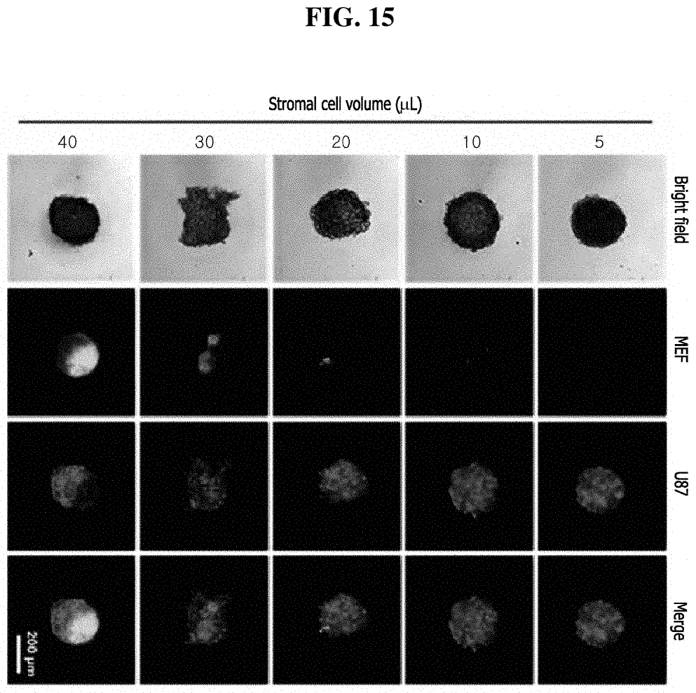

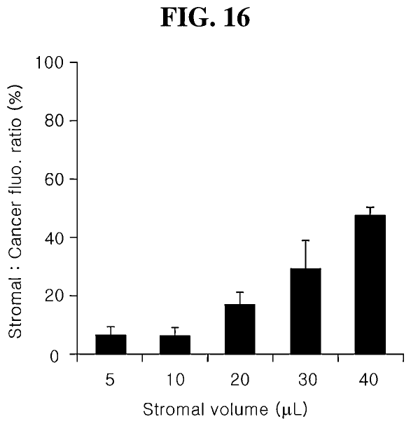

FIG. 14 is a schematic flowchart of another example of a spheroid culturing method according to an embodiment of the present invention, FIG. 15 is a schematic diagram of stromal cells and cancer cells in a spheroid culturing method according to an embodiment of the present invention, and FIG. 16 is a graph schematically showing a relationship between an injected amount of stromal cells and cancer cells in a spheroid culturing method according to an embodiment of the present invention.

Referring to FIGS. 14 to 16, a spheroid generator 10 in which the diameter of a sub-injection part 18 is increased from one side to the other side of a culture plate 120 is disposed (S21). In this case, main injection parts 14 having the same size are formed in all of the spheroid generators 10.

Cancer cells are labeled with a first fluorescent solution (S22). As the first fluorescent solution, red fluorescent protein is suggested.

A cancer cell solution 21 is prepared (S23). That is, the cancer cell solution 21 is prepared by mixing a plurality of cancer cells with a culture medium 24.

The cancer cell solution 21 is injected into main injection parts 14 of the plurality of spheroid generators 10 (S24). In this case, since main injection parts 14 having the same size are formed in all of the spheroid generators 10, the same amount of the cancer cell solution 21 is injected into all of the main injection parts 14.

As the cancer cells of the cancer cell solution 21 are cultured in the plurality of spheroid generators 10, a spheroid 25 is formed (S25). In this case, the step of forming a spheroid 25 as the cancer cells of the cancer cell solution 21 are cultured in the plurality of spheroid generators 10 is performed for 12 to 36 hours.

Stromal cells are labeled with a second fluorescent solution (S26). As the second fluorescent solution, green fluorescent protein is suggested.

A stromal cell solution is prepared (S27). That is, the stromal cell solution is prepared by mixing a plurality of stromal cells with a culture medium 24.

The stromal cell solution is injected into a sub-injection part 18 of each of the plurality of spheroid generators 10 (S28). In this case, since the size of the sub-injection part 18 is increased from one side to the other side of a culture plate 120, an input amount (volume) of the stromal cell solution 27 is increased from the sub-injection part 18 at one side of the culture plate to the sub-injection part 18 at the other side.

In the plurality of spheroid generators 10, a spheroid 25 in which the proportions of cancer cells and stromal cells are different according to an injected amount of the stromal cell solution 27 is cultured (S29). That is, when an injected amount of the stromal cell solution 27 is small, a ratio of stromal cells to cancer cells in the spheroid 25 is decreased. Also, when an injected amount of the stromal cell solution 27 is large, a ratio of stromal cells to cancer cells in the spheroid 25 is increased.

A culture medium 24 is injected into the main injection part 14 of each of the plurality of spheroid generators 10 so that the spheroid 25 drops from the main injection part 14 (S30). In this case, the spheroid 25 drops from the main injection part 14 toward one side due to the injection pressure of the culture medium 24 injected into the main injection part 14.

It is possible to check a ratio of cancer cells and stromal cells according to an input amount of the stromal cell solution 27 (S31). In this case, a cancer cell proportion of the spheroid 25 can be confirmed using a microscope.

Referring to FIGS. 15 to 16, the volume of stromal cells is shown in the MEF line, the volume of cancer cells is shown in the U87 line, and the mixed volume of stromal cells and cancer cells is shown in the Merge line. It can be seen that a stromal cell proportion increases as an injected amount of stromal cells increases.

Although the present invention has been described with reference to exemplary embodiments illustrated in the drawings, the above-described descriptions are only exemplary. Therefore, it should be understood by those skilled in the art that various modifications and equivalent other exemplary embodiments are possible from the above-described descriptions.

Therefore, the true technical protection scope of the present invention should be defined by following claims.

1. A spheroid generator comprising:
a body part;
a main injection part which is formed in the body part so that a cancer cell solution is injected and configured to stop the flow of the cancer cell solution when the cancer cell solution comes into contact with a culture medium; and
a sub-injection part which is formed in the body part so that an anticancer substance or a stromal cell solution is injected, and communicates with the main injection part so that the injected anticancer substance or stromal cell solution flows to the main injection part.

2. The spheroid generator of claim 1, wherein the main injection part includes:
a main inlet part into which the cancer cell solution is injected and which communicates with the sub-injection part; and
a main tube which communicates with the main inlet part and is configured to stop the flow of the cancer cell solution when the cancer cell solution comes into contact with the culture medium.

3. The spheroid generator of claim 2, wherein the main tube includes:
a first main tube which is connected to one side of the main inlet part and formed to have an inner diameter smaller than that of the main inlet part; and
a second main tube which is connected to one side of the first main tube and formed to have a diameter larger than that of the first main tube.

4. The spheroid generator of claim 3, wherein the body part further includes a communication channel part formed so that one side of the main inlet part and one side of the sub-injection part communicate with each other.

5. The spheroid generator of claim 1, wherein one sub-injection part is formed around the main inlet part.

6. The spheroid generator of claim 1, wherein a plurality of sub-injection parts is formed around the main inlet part.

7. The spheroid generator of claim 6, wherein the plurality of sub-injection parts is formed to have the same inner diameter.

8. The spheroid generator of claim 6, wherein the plurality of sub-injection parts is formed to have different inner diameters.

9. The spheroid generator of claim 1, wherein the body part, the main injection part, and the sub-injection part are manufactured by 3D printing.

10. A spheroid culture kit comprising:
a culture tank in which a culture medium is accommodated;
a culture plate which is disposed at one side of the culture tank and in which a plurality of culture chambers is formed; and
a plurality of spheroid generators which are respectively disposed in the plurality of culture chambers,
wherein the spheroid generator is the spheroid generator described in claim 1.

11. A method of culturing a spheroid comprising:
disposing a spheroid generator in which the size of a sub-injection part is increased from one side to the other side of a culture plate;
injecting a cancer cell solution into a main injection part of each of a plurality of spheroid generators;
forming a spheroid as cancer cells of the cancer cell solution are cultured in the plurality of spheroid generators;
injecting an anticancer substance into the sub-injection part of each of the plurality of spheroid generators;
changing a cancer cell proportion of the spheroid according to an input amount of the anticancer substance in the plurality of spheroid generators; and
injecting a culture medium into the main injection part of each of the plurality of spheroid generators so that the spheroid drops from the main injection part.

12. The method of claim 11, wherein the step of forming a spheroid as cancer cells of the cancer cell solution are cultured in the plurality of spheroid generators is performed for 12 to 36 hours.

13. The method of claim 11, wherein the step of changing a cancer cell proportion of the spheroid according to the anticancer substance is performed for 12 to 36 hours.

14. A method of culturing a spheroid comprising:
disposing a spheroid generator in which the diameter of a sub-injection part is increased from one side to the other side of a culture plate;
injecting a cancer cell solution into a main injection part of each of a plurality of spheroid generators;
forming a spheroid as cancer cells of the cancer cell solution are cultured in the plurality of spheroid generators;
injecting a stromal cell solution into the sub-injection part of each of the plurality of spheroid generators;
culturing a spheroid in which the proportions of the cancer cell and stromal cell are different according to an injected amount of the stromal cell solution in the plurality of spheroid generators; and
injecting a culture medium into the main injection part of each of the plurality of spheroid generators so that the spheroid drops from the main injection part.

15. The method of claim 14, wherein the cancer cell solution includes a cancer cell labeled with a first fluorescent solution.

16. The method of claim 15, wherein the stromal cell solution includes a stromal cell labeled with a second fluorescent solution.
